(19) United States
(12) Patent Application Publication
(10) Pub. No.: US 2009/0258098 A1
(43) Pub. Date: Oct. 15, 2009

(54) PENETRATING CARRIER, ANTIFUNGAL COMPOSITION USING THE SAME AND METHOD FOR TREATMENT OF DERMATOPHYTE INFECTIONS

(76) Inventors: LANE ROLLING, Las Vegas, NV (US); Craig Oberg, Eden, UT (US); Evan Call, Bountiful, UT (US)

Correspondence Address: David B. Tingey, 1800 Eagle Gate Tower, 60 East South Temple, Salt Lake City, UT, 84145, US

(21) Appl. No.: 12/423,797

(22) Filed: Apr. 14, 2009

Related U.S. Application Data
(60) Provisional application No. 61/045,246, filed on Apr. 15, 2008.

(51) Int. Cl. A61K 36/00; A61P 31/10; A61K 31/11; A61K 31/05; A61K 31/085

(52) U.S. Cl. 424/773; 424/725; 424/774; 424/775; 424/776; 424/777; 424/778; 424/779; 514/701; 514/731; 514/703; 514/720

(57) ABSTRACT

An antifungal composition and penetrating carrier system for topical treatment of dermatophytic infection and secondary bacterial infections. The antifungal composition includes various fungistatic and fungicidal essential oil components, or combinations thereof. The penetrating carrier system may include various ingredients, including a penetration enhancer, such as isopropyl myristate.

### RELATED APPLICATIONS

The present application claims priority to U.S. Provisional Patent Application No. 61/045,246 entitled “PENETRATING CARRIER, ANTIFUNGAL COMPOSITION USING THE SAME AND METHODS FOR TREATMENT OF DERMATOPHYTE INFECTIONS” which was filed Apr. 15, 2008, and is incorporated herein in its entirety.

### BACKGROUND OF THE INVENTION

The present disclosure relates to treatment of dermatophyte infections and associated bacterial infections. Specifically, this disclosure relates to the delivery of essential oils, as well as specific components of essential oils, for the treatment of human dermatophytic infections.

For centuries plant extracts have been used as food preservatives. These plant elements and extracts have also been used for medicinal purposes to prevent infections by inhibiting the growth of pathogenic microorganisms. The plant's inhibitory characteristics are largely due to essential oils of the plant. Often, these essential oils contain chemicals with inhibitory properties towards bacteria, viruses, and fungi. Essential oils are naturally occurring mixtures of terpines, alcohols, aldehydes, ketones, carboxylic acids, esters, lactones, and sulfides synthesized by plants. Essential oil components are commonly found in the fruit, flowers, roots, leaves, and bark of plants. Compounds in essential oils are used by the plant to attract or repulse insects, and to protect against a wide spectrum of disease-causing agents. Additionally, naturally synthesized essential oils have been in contact with humans for centuries in their ingested form as a component of food additives (i.e.: spices and herbs) used as seasonings and preservatives. As a result of this long term interaction, most essential oils and many of their components are generally recognized as safe (GRAS) materials.

Although certain essential oils have been shown effective in treating dematophytic infections, there remains an inability to effectively deliver active components of the essential oils to infected tissue. Thus, while techniques currently exist that are used to deliver and apply certain essential oils to infected tissues, challenges still exist. Accordingly, it would be an improvement in the art to augment or even replace current techniques with other techniques.

### BRIEF SUMMARY OF THE INVENTION

The present disclosure relates to treatment of dermatophyte infections and associated bacterial infections. Specifically, this disclosure relates to the delivery of essential oils, as well as specific components of essential oils, for the treatment of human dematophytic infections.

Accordingly, one object of the present invention is to provide a carrier for topical medicaments that can penetrate and transport the medicament into the keratinized layers of a patient's skin tissue.

A further object of the present invention is to provide a topical composition for treatment of fungal infections using this carrier.

A further object of the present invention is to provide a topical antifungal composition effective at treating Candida albicans (C. albicans), Epidermophyton floccosum (E. floccosum), Microsporum gypseum (M. gypseum), Trichophyton metagrophytes (T. metagrophytes), and Trichophyton rubrum (T. rubrum).

A further object of the present invention is to provide a topical antifungal composition effective at treating secondary pathogenic bacteria including Escherichia coli (E. coli), Pseudomonas aeruginosa (P. aeruginosa), Staphylococcus aureus (S. aureus), Staphylococcus sanguis (S. sanguis), and Methicillin-resistant staphylococcus aureus (MSRA).

A further object of the present invention is to provide a topical antifungal composition that contains only GRAS ingredients, and requires no debridement during treatment.

A further object of the present invention is to provide a topic treatment that is fungicidal and fungistatic against common human dermatophytic fungi.

A further object of the present invention is to provide an antifungal composition in which the active ingredients are thermostable.

A further object of the present invention is to provide a method for the topical treatment of dermatophytic infection using such antifungal composition.

These and other objects of the present invention have been satisfied by the discovery of a penetrating carrier system comprising sodium stearoyl lactylate and isopropyl myristate, and optionally aloe vera concentrate, disodium EDTA, carbomer, propylene glycol, glycerine, methylparaben, propylparaben, persea gratissima, prumus amygdalus dulcis, glyceryl sterate, cetearyl alcohol, tocopherol, tocopherol acetate, dimethicone, triethanolamine, sodium hydroxide, sandal wood oil, and lavender oil, and its use to prepare an antifungal composition comprising: an effective antifungal amount of a mixture of antifungal essential oils, and the penetrating carrier system, wherein the mixture of antifungal essential oils comprises effective amounts of one or more essential oils selected from the group comprising juniper berry, cinnamaldehyde, carvacrol, citral, eugenol, methyl eugenol, and thymol.

### DETAILED DESCRIPTION OF THE INVENTION

The present disclosure relates to treatment of dermatophyte infections and associated bacterial infections. Specifically, this disclosure relates to the delivery of essential oils, as well as specific components of essential oils, for the treatment of human dematophytic infections.

Throughout this specification, there are ranges defined by upper and lower boundaries. Each lower boundary can be combined with each upper boundary to define a range. The lower and upper boundaries should each be taken as a separate element.

The penetrating carrier system of the present invention comprises at least one antifungal essential oil component, isopropyl myristate, and an emulsifier. For example, in one embodiment the emulsifier is sodium stearoyl lactylate. Preferably, the range of the ratio of the amount by weight of the isopropyl myristate to the amount by weight of the sodium stearoyl lactylate has an upper boundary of approximately 10:1. Examples of other upper boundaries include about 1; 75:1; 50:1; 30:1; 20:1; 15:1; 13:1; 21:1; 4:1; 3:1; and 2:1.

Preferably, the range of the ratios of the amount by weight of the isopropyl myristate to the amount by weight of the sodium stearoyl lactylate has a lower boundary of approximately 1:200. Examples of other lower boundaries include about 1:400; 1:300; 1:250; 1:175; 1:150; 1:125; 1:100; 1:75; 1:50; 1:30; 1:20; 1:15; 1:13; 1:12, 1:3; 1:2; and 1:1.

The isopropyl myristate of the penetrating carrier system acts as a penetration enhancer to increase the permeability of the skin to the antifungal composition. A penetration enhancer or permeation enhancer is an agent used to increase the permeability of the skin to a pharmacologically active agent to increase the rate at which the drug diffuses through the skin and enters the tissues and bloodstream. A chemical skin penetration enhancer increases skin permeability by reversibly altering the physiochemical nature of the stratum corneum to reduce its diffusional resistance. In a review of the technical and patent literature up to 1996, more than 275 different chemical compounds were found to be cited as skin penetration enhancers. Most of the compounds are GRAS ingredients that would often be considered inert by a formulator. Osborne D W, Henke J J, Pharmaceutical Technology, November. 1997, pp 58-86. The compounds cited in the article are incorporated herein by reference.

Therefore, one of skill in the art will appreciate that other penetration enhancer may be substituted for the isopropyl myristate within the scope of this invention. Examples of additional penetration enhancers include: alcohols, such as ethanol and isopropanol, polyols, such as n-alkanols, limonene, terpenes, dioxolane, propylene glycol, ethylene glycol, other glycols, and glycerol; sulfoxides, such as dimethylsulfoxide (DMSO), dimethylformanide, methyl dodecyl sulfoxide, dimethylacetamide; esters, such as isopropyl palmitate, ethyl acetate, butyl acetate, methyl proprionate, capric/caprylic triglycerides, ketones; amides, such as acetamide oleates such as triolein; various surfactants, such as sodium lauryl sulfate; various alkanoic acids such as caprylic acid; lactam compounds, such as azone; alkanols, such as oleyl alcohol; dialkylamino acetates, and admixtures thereof.

The emulsifier used in the penetrating carrier system can be any emulsifying agent. For the purposes of this specification, an emulsifying agent includes any additive that promotes the formation of a stable mixture, or emulsion, of oil and water. Common emulsifiers include metallic soaps, certain animal and vegetable oils, and various polar compounds. Examples of emulsifying agents suitable for use in the penetrating carrier system include cera alba; cera flava; carbomer; glycerol stearate; emulsifying wax NF; cetaryl alcohol; PEG-20 stearate; cetyl alcohol; propylene glycol; stearyl alcohol NF; polysorbate 80; and lecithin.

In some embodiments of the invention, the penetrating carrier system can further comprise at least one of an emollient, a humectant, an additional emulsifier, a preservative, a skin aid, a conditioner, a chelating agent, a rheology modifier, a moisturizer, a scent, a diluent, or mixtures thereof.

Up to approximately 5 wt %, 10 wt %, 20 wt %, 30 wt %, 40 wt %, 50 wt %, 60 wt %, 70 wt %, 80 wt %, 90 wt %, or 97 wt % of the emulsifier can be replaced with an emollient, a humectant, an additional emulsifier, a preservative, a skin aid, a conditioner, a chelating agent, a rheology modifier, a moisturizer, a scent, a diluent, or mixtures thereof. For example, in a penetrating carrier system comprising isopropyl myristate and sodium stearoyl lactylate, preferably about 97 wt % of the sodium stearoyl lactylate is replaced with at least one of an emollient, a humectant, an additional emulsifier, a preservative, a skin aid, a conditioner, a chelating agent, a rheology modifier, a moisturizer, a scent, a diluent, a pH titrating agent, or mixtures thereof.

Examples of preferred emollients include persea gratissima at about 2.3 wt %, prunus amygdalus dulcis at about 2.3 wt %, and dimethicone at about 0.5 wt %. Examples of preferred humectants include propylene glycol at about 0.8 wt %, and glycerine at about 4.5 wt %. Examples of preferred additional emulsifiers include glycerol stearate at about 1.5 wt %. Examples of preferred preservatives include methyparaben at about 0.2 wt %, and propylparaben at about 0.1 wt %. Examples of preferred skin aids include tocopherol at about 0.5 wt %, and tocopherol acetate at about 0.2 wt %. An example of a preferred conditioner is cetearyl alcohol at about 0.5 wt %. An example of a preferred chelating agent is disodium EDTA at about 0.1 wt %. An example of a rheology modifier is carbomer at about 0.3 wt %. An example of a moisturizer is aloe vera concentrate at about 2.7 wt %. Examples of scents include sandal wood oil at about 0.9 wt %, and lavender oil at about 1.0 wt %. An example of a diluent includes distilled de-ionized (DDI) water at about 77.6 wt %. Finally, an example of a pH titrating agent includes triethanolamine (99%) at about 0.5 wt %. Additional components of the carrier system include antifungal essential oils, as discussed in detail below.

Suitable antifungal essential oils can be manufactured (i.e., synthesized or partially synthesized). Alternatively, the essential oil can be obtained from a plant or plant component (e.g., plant tissue). Suitable plant or plant components include, e.g., a herb, flower, fruit, seed, bark, stem, root, nettle, bulb, berry, rhizome, rootstock, leaf, or a combination thereof.

Any suitable essential oil can be employed provided (1) the essential oil has therapeutic properties (e.g., the essential oil has antifungal properties), (2) the essential oil remains thermally stable in the composition, and (3) the essential oil is non-toxic to mammals (e.g., humans) and will be suitable for topical medicinal use. Preferably, the thermostability of the essential oil is over a prolonged period of time, e.g., up to about 3 years, up to about 1 year, or up to about 6 months, typically experienced in the manufacturing, packaging, shipping, and/or storage of the composition. The preferred essential oil will also preferably comply with any controlling or governing body of law.

Suitable specific essential oils include one or more of the following: ajowan, sweet almond oil, allspice, aloe vera oil, ammi visnaga (khella), amyris, angelica root, angelica seed, anise, anise seed, star anise, apricot kernel oil, absolute arnica, avocado oil, unrefined avocado oil, Copaiba balsam, balsam Peru genuine, balsam Peru oil, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot oil, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel oil, clove, clove bud, clove leaf, cocoa, cocoa butter, unrefined cocoa butter, coconut oil, refined coconut oil, cognac, combava petitgrain, coriander, green coriander, cornmint, costus oil, cumin, cypress, davana oil, dill, dill weed, elemi, erigeron (fleabane), eucalyptus citriodora, eucalyptus globulus, lemon eucalyptus, fennel, sweet fennel, fenugreek, fir, Canada fir needle, Siberia fir needle, white fir needle, frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed oil, hazelnut oil, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper berry, Siberia juniper berry, Croatia juniper berry, lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, olibanum, absolute opopanax, bitter orange, blood orange, sweet orange, wild West Indian orange, oregano, orris root, concrete orris, osmanthus, palm oil, refined palm oil, palmarosa, paprika, parsley seed, patchouli, Indian patchouli oil, Indonesian patchouli oil, peanut, peanut oil, pecan oil, pennyroyal, pepper, black pepper, super black pepper, peppermint, India peppermint, USA baby mint peppermint, pet perfume, petitgrain (orange leaves), white pine, pine needle, evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce, St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra.

Specifically, suitable exemplary essential oils include cinnamaldehyde, juniper berry, carvacrol, citral, eugenol, methyl eugenol, thymol, or a combination thereof. Preferably, in order to minimize the build-up of resistance to the ingredients, combinations of different essential oils can be used in the penetrating carrier system. For example, in one embodiment the penetrating carrier system comprises two essential oils. In another embodiment, the penetrating carrier system comprises three or more essential oils.

The amount of essential oils in the penetrating carrier system is within the effective ranges of the individual oils. For example, in one embodiment a single essential oil is added to the system within an effective range of 500 ppm to 3500 ppm. In another embodiment, a first essential oil is added to the system within an effective range of 500 ppm to 3500 ppm, and a second essential oil is added to the system within an effective range of 100 ppm to 1500 ppm. In another embodiment, a first essential oils is added to the system within an effective range of 500 ppm to 3500 ppm, a second essential oil is added to the system within an effective range of 50 ppm to 1500 ppm, and a third essential oil is added to the system within an effective range of 50 ppm to 1500 ppm. In another embodiment, a first and second essential oil is added to the system within an effective range of 500 ppm to 3500 ppm, and a third essential oil is added to the system within an effective range of 50 ppm to 1500 ppm. In yet another embodiment, a first, a second, and a third essential oil is added to the system within an effective range of 500 ppm to 3500 ppm, and a fourth essential oil is added to the system within an effective range of 50 ppm to 1500 ppm. Finally, in yet another embodiment, one or more essential oils are added to the system within an effective range of 500 ppm to 3500 ppm, and one or more additional essential oils are added to the system within an effective range of 50 ppm to 1500 ppm.

The penetrating carrier system may be applied topically to a patient by any effective carrying agent. For example, in one embodiment the penetrating carrier system is incorporated into an aerosol spray. In this embodiment, a user applies the antifungal composition to the dermatophytic infection by applying the aerosol spray directly to the infected tissue. In another embodiment, the penetrating carrier system is incorporated into a talcum powder. In this embodiment, a user applies the antifungal composition to the dermatophytic infection by applying the talcum powder directly to the infected tissue. In yet another embodiment, the penetrating carrier system is incorporated into at least one of an ointment, a lotion, a cream, a shoe insole, a gel and a fabric. In this embodiment, a user applies the antifungal composition to the dermatophytic infection by maintaining contact between the infected tissue and the carrying agent. Finally, in another embodiment the penetrating carrier system is incorporated into an aqueous solution that may be sprayed directly onto the infected tissue, or may the infected tissue may be soaked directly in the aqueous solution.

The penetrating carrier system is effective as a fungistatic, as well as a fungicidal for a wide variety of common human dematophytic fungi. Additionally, the penetrating carrier system is effective as an antibacterial agent to inhibit or prevent secondary bacterial infections that can follow a dermatophytic infection. For example, Table 3 (see below) demonstrates non-limiting effective amounts of preferred essential oils as both fungistatic and fungicidal agents for selected human dermatophytes. Furthermore, Table 2 (discussed in detail below) demonstrates non-limiting effective amounts of cinnamaldehyde as an antibacterial agent for selected pathogenic bacterium. For example, delivery systems containing thymol and cinnamaldehyde prove to be fungicidal against all tested dermatophytes. Additionally, the cinnamaldehyde-based delivery system also inhibits E. coli, P. aeruginosa, S. aureus, S. sanguis, and MSRA. One of skill in the art will appreciate that tables 2 and 3 are provided as non-limiting examples and that the antifungal essential oils may provide additional antifungal and antibacterial benefits to a wide range of fungi and bacterium strains.

The penetrating carrier system of the present invention can be produced by a variety of methods. Preferably, the one or more essential oils and the isopropyl myristate are preformulated in separate solutions. Thereafter, the components are mixed in effective ratios, as discussed above. Any optionally added ingredients, such as a moisturizer, a chelating agent, a humectant, a rheology modifier, a preservative, an emollient, an additional emulsifier, a conditioner, a skin aid, or a scent, are preferably added according to the ratios discussed above.

For example, one method of producing the penetrating carrier system includes first adding the chelating agent (disodium EDTA) to the diluent (DDI water) and mixing until dissolved. Next, the rheology modifier (carbomer) is dispersed on the surface of the chelating agent and the diluent mixture. After the rheology modifier has fully wetted, the solution is then mixed as a slow speed, thereby yielding a first solution. The humectants (propylene glycol and glycerin) and the preservatives (methylparaben and propylparaben) are then mixed separately and added to the first solution. The combined solutions are then heated to 65° C. The emollients (persea gratissima, prunus amygdalus dulcis, and dimethicone), the emulsifiers (sodium stearoyl lactylate and glycerol stearate), the conditioner (cetearyl alcohol), the skin aids (tocopherol and tocopherol acetate), the penetration enhancer (isopropyl myristate), and the one or more essential oils are then combined, heated to 65° C., and mixed slightly to provide a second solution. The second solution is then combined with the first solution. The titrating agent (triethanolamine (99%)) is then added to the mixture, along with sodium hydroxide to achieve a pH between 6.9 and 7.2. Additionally, the scents (sandal wood oil and lavender oil) are added during the titration step. Finally, the emulsion is mixed with moderate agitation until the temperature reaches 40° C.

### EXAMPLES

### Microbial Testing

Various embodiments of the penetrating carrier system of the present invention were tested for their antimicrobial/biostatic potential by a laboratory test method, which provides a qualitative and semi-qualitative procedure for the evaluation of antimicrobial activity by disk assay. All dermatophytic fungal strains (Table 1) were grown on modified Sabouraud agar (pH adjusted to 7). The cultures were grown for one week previous to the experiments at 30° C., while stock culture plates were maintained at 4° C. until needed. In order to determine the most effective essential oil components, fungal cultures were streaked three ways across the surface of the agar and a sterile 6 mm paper disk was placed in the center of the plate. The sterile disk was then loaded with 500 ppm, 1000 ppm, 1500 ppm or 3000 ppm of the test compound. A control was provided by placing 2 ul of alcohol on plates that were inoculated for this purpose. The plates were incubated for seven (7) days at 30° C. The zone of inhibition was measured around each loaded disk in four directions. The average measurement was then determined and recorded in millimeters as the zone of inhibition.

### Example 1

### Penetrating Carrier System

The penetrating carrier system was developed to facilitate the testing of plant oil components in vivo and to determine their potential as remedies for both dermatophytic, and bacterial infections. The essential plant oil components cinnamaldehyde and thymol were individually incorporated into the lotion-based penetrating carrier system at different concentrations. Each system was then tested for their potential to inhibit C. albicans, E. floccosum, M. gypseum, T. mentagrophytes, and T. rubrum using the disk assay method. All components remained uniformly suspended within the lotion, despite being primarily water-based. Thymol-containing lotion proved to fungicidal against all four dermatophytes at 1000 ppm while the cinnamaldehyde-containing lotion was inhibitory at 1500 ppm (see Table 1). The zone of inhibition was significantly larger for the 1500 ppm cinnamaldehyde lotion than for the 1000 ppm thymol lotion. The cinnamaldehyde-based lotion was also tested against E. coli, P. aeruginosa, S. aureus, S. sanguis, and MSRA. A 1500 ppm cinnamaldehyde lotion inhibited all the test bacteria except P. aeruginosa (see Table 2).

TABLE 1

| | E. floccosum | | | M. gypseum | | | T. mentagrophytes | | | T. rubrum | | |
|---|---|---|---|---|---|---|---|---|---|---|---|---|
| Lotion | Zone | Ave | SD | Zone | Ave | SD | Zone | Ave | SD | Zone | Ave | SD |
| Control | 0 | 0.00 | N/A | 0 | 0.00 | N/A | 0 | 0.00 | N/A | 0 | 0.00 | N/A |
| Thymol | 2 | 6.67 | 4.16 | 2 | 1.67 | 0.58 | 1 | 1.67 | 1.15 | 4 | 2.67 | 1.15 |
| 1000 ppm | 10 | | | 1 | | | 1 | | | 2 | | |
| | 8 | | | 2 | | | 3 | | | 2 | | |
| Thymol | 10 | 9.67 | 2.52 | 11 | 10.00 | 2.65 | 5 | 3.67 | 1.15 | 6 | 3.33 | 2.52 |
| 1500 ppm | 12 | | | 7 | | | 3 | | | 1 | | |
| | 7 | | | 12 | | | 3 | | | 3 | | |
| Cinnamaldehyde | 22 | 24.00 | 1.73 | 22 | 22.33 | 1.53 | 9 | 8.67 | 0.58 | 20 | 18.33 | 2.89 |
| 1500 ppm | 25 | | | 21 | | | 9 | | | 15 | | |
| | 25 | | | 24 | | | 8 | | | 20 | | |
| Cinnamaldehyde | 35 | 30.33 | 5.69 | 31 | 31.00 | 2.00 | 20 | 21.67 | 2.08 | 28 | 26.67 | 1.53 |
| 3000 ppm | 32 | | | 29 | | | 21 | | | 27 | | |
| | 24 | | | 33 | | | 24 | | | 25 | | |

TABLE 2

| Lotion | E. coli | | | P. aeruginosa | | | S. aureus | | | S. sanguis | | | MRSA | | |
|---|---|---|---|---|---|---|---|---|---|---|---|---|---|---|---|
| Conc. | Zone* | Ave | SD | Zone | Ave | SD | Zone | Ave | SD | Zone | Ave | SD | Zone | Ave | SD |
| 1500 ppm | 3 | 2.33 | 0.76 | 0 | 0.00 | 0.00 | 10 | 9.67 | 0.58 | 1 | 2.00 | 2.65 | 15 | 12.33 | 2.52 |
| | 2.5 | | | 0 | | | 9 | | | 0 | | | 10 | | |
| | 1.5 | | | 0 | | | 10 | | | 5 | | | 12 | | |
| 3000 ppm | 6 | 6.67 | 1.15 | 1 | 0.67 | 0.58 | 13 | 13.33 | 0.58 | 9 | 5.67 | 4.93 | 15 | 15.33 | 1.53 |
| | 6 | | | 0 | | | 14 | | | 8 | | | 17 | | |
| | 8 | | | 1 | | | 13 | | | 0 | | | 14 | | |

*Zones are measured in mm.

### Example 2

### Minimum Inhibitory Concentration (MIC)

The MIC for the seven most inhibitory essential oil components from the disk assay (i.e.: carvacrol, cinnamaldehyde, citral, eugenol, methyl eugenol, thymol, and juniper berry) were then determined using a poison media procedure. Each individual essential oil component was mixed with molten modified Sabouraud agar in 100 ppm, 500 ppm, or 1000 ppm (v/v). The poison media agar was then poured and allowed to cool for at least 48 hours. Separate cultures of four dermatophytes (i.e.: E. floccosum, M. gypseum, T. mentagrophytes, and T. rubrum) were grown on modified Sabouraud agar for four days until a confluent mat of mycelium developed. The four cultures were then cut into 8 mm plugs and placed upside down with the fungi culture on the surface of the poison media. This procedure was run in triplicate. In addition, several mixtures containing various combinations of the essential oil components were tested using the same procedure. The plates were incubated for one week at 30° C. Plates were then examined for growth. Plates with no visible growth after one week on the poison media were listed as fungistatic

Plugs showing no growth on the poison media were then transferred to modified SDA, allowed to incubate for an additional week at 30° C., and then reexamined for growth. All components were run in triplicate. Plates with no growth after the additional week were listed as fungicidal.

All of the tested components were able to inhibit the growth of the majority of the tested microbes at 1000 ppm (see Table 3). The most effective of the chemicals were carvacrol and thymol, which were able to inhibit the growth of several organisms at 100 ppm. All of the component mixtures were very effective against the test fungi, and were mostly fungicidal at 500 ppm.

TABLE 3

| | Epidermophyton | Microsporum | Trichophyton | Trichopyton |
|---|---|---|---|---|
| | floccosum | gypseum | mentagrophytes | rubrum |
| Carvacrol | | | | |
| 100 ppm | | fungistatic | | fungistatic |
| 500 ppm | fungicidal | fungicidal | fungicidal | fungicidal |
| 1000 ppm | fungicidal | fungicidal | fungicidal | fungicidal |
| Cinnamaldehyde | | | | |
| 100 ppm | | | | |
| 500 ppm | fungicidal | fungicidal | | fungicidal |
| 1000 ppm | fungicidal | fungicidal | fungicidal | fungicidal |
| Citral | | | | |
| 100 ppm | | | | |
| 500 ppm | | | | |
| 1000 ppm | | fungicidal | fungistatic | fungicidal |
| Eugenol | | | | |
| 100 ppm | | | | |
| 500 ppm | fungicidal | fungicidal | fungistatic | fungistatic |
| 1000 ppm | fungicidal | fungicidal | fungistatic | fungicidal |
| Methyl Eugenol | | | | |
| 100 ppm | | | | |
| 500 ppm | fungistatic | fungistatic | fungistatic | fungistatic |
| 1000 ppm | fungistatic | fungistatic | fungistatic | fungistatic |
| Thymol | | | | |
| 100 ppm | fungistatic | fungistatic | fungistatic | fungicidal |
| 500 ppm | fungicidal | fungicidal | fungicidal | fungicidal |
| 1000 ppm | fungicidal | fungicidal | fungicidal | fungicidal |
| Carvacrol/Eugenol | | | | |
| 100 ppm | | | | |
| 500 ppm | fungicidal | fungicidal | fungicidal | fungicidal |
| 1000 ppm | fungicidal | fungicidal | fungicidal | fungicidal |
| Eugenol/Cinnam. | | | | |
| 100 ppm | | | | |
| 500 ppm | fungicidal | fungicidal | fungistatic | fungistatic |
| 1000 ppm | fungicidal | fungicidal | fungicidal | fungicidal |
| Thymol/Carvacrol | | | | |
| 100 ppm | fungistatic | fungistatic | fungistatic | fungistatic |
| 500 ppm | fungicidal | fungicidal | fungicidal | fungicidal |
| 1000 ppm | fungicidal | fungicidal | fungicidal | fungicidal |
| Thymol/Cinnam. | | | | |
| 100 ppm | | | | |
| 500 ppm | fungicidal | fungicidal | fungicidal | fungicidal |
| 1000 ppm | fungicidal | fungicidal | fungicidal | fungicidal |
| Carvacrol/Cinnam. | | | | |
| 100 ppm | fungicidal | | | |
| 500 ppm | fungicidal | fungicidal | fungicidal | fungicidal |
| 1000 ppm | fungicidal | fungicidal | fungicidal | fungicidal |
| Eugenol/Thymol | | | | |
| 100 ppm | fungistatic | | fungistatic | fungistatic |
| 500 ppm | fungicidal | fungicidal | fungicidal | fungicidal |
| 1000 ppm | fungicidal | fungicidal | fungicidal | fungicidal |

* a blank space indicates fungal growth was observed on the Petri plate

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed is:

1. A composition for treating a dermatophytic infection, comprising:
an excipient;
a penetration enhancer; and
at least one antifungal essential oil component.

2. The composition of claim 1, wherein the penetration enhancer is selected from the group comprising:
(i) isopropyl myristate;
(ii) an alcohol;
(iii) an n-alkanol;
(iv) a sulfoxide;
(v) an ester;
(vi) an amide;
(vii) a surfactant;
(viii) an alkanoic acid;
(ix) a lactam compound;
(x) an alkanol; and
(xi) a dialkylamino acetate.

3. The composition of claim 1, further comprising an emulsifying agent selected from the group comprising:
(i) cera alba;
(ii) cera flava;
(iii) carbomer;
(iv) glycerol stearate;
(v) emulsifying wax NF;
(vi) cetaryl alcohol;
(vii) PEG-20 stearate;
(viii) cetyl alcohol;
(ix) propylene glycol;
(x) stearyl alcohol NF;
(xi) polysorbate 80; and
(xii) lecithin.

4. The composition of claim 1, further comprising a skin conditioner selected from the group comprising:
(i) an emollient;
(ii) a humectants;
(iii) an emulsifier;
(iv) a preservative;
(v) a skin aid;
(vi) a conditioner;
(vii) a chelating agent;
(viii) a rheology modifier;
(ix) a moisturizer;
(x) a scent; and
(xi) a diluent.

5. The composition of claim 1, wherein the antifungal essential oil component is an essential oil derived from at least one of:
(i) an herb;
(ii) a flower;
(iii) a fruit;
(iv) a seed;
(v) a bark;
(vi) a stem;
(vii) a root;
(viii) a nettle;
(ix) a bulb;
(x) a berry;
(xi) a rhizome;
(xii) a rootstock; and
(xiii) a leaf.

6. The composition of claim 5, wherein the antifungal essential oil component is at least one of:
(i) cinnamaldehyde;
(ii) juniper berry;
(iii) carvacrol;
(iv) citral;
(v) eugenol;
(vi) methyl eugenol; and
(vii) thymol.

7. The composition of claim 1, further comprising a first essential oil within an effective range from about 500 ppm to about 3500 ppm.

8. The composition of claim 7, further comprising a second essential oil within an effective range from about 500 ppm to about 3500 ppm.

9. The composition of claim 7, further comprising a second essential oil within an effective range from about 100 ppm to about 1500 ppm.

10. The composition of claim 1, further comprising a first group of essential oils, each component oil of the first group being added to the composition within an effective range from about 500 ppm to about 3500 ppm, and a second group of essential oils, each component oil of the second group being added to the composition within an effective range from about 50 ppm to about 1500 ppm.

11. The composition of claim 1, wherein the excipients is selected from a group comprising:
(i) an aerosol;
(ii) a talcum powder;
(iii) an aqueous solution;
(iv) an ointment;
(v) a lotion;
(vi) a cream;
(vii) a shoe insole;
(viii) a gel; and
(ix) a fabric.

12. A method for treatment of fungal infection comprising topically applying to a treatment area an effective amount of liquid composition which comprises a carrier system having an excipient, a penetration enhancer, at least one antifungal essential oil selected from the group comprising:
(i) cinnamaldehyde;
(ii) juniper;
(iii) thymol;
(iv) citral;
(v) eugenol; and
(vi) carvacrol.

13. The method of claim 12, wherein the penetration enhancer is selected from the group comprising:
(i) isopropyl myristate;
(ii) an alcohol;
(iii) an n-alkanol;
(iv) a sulfoxide;
(v) an ester;
(vi) an amide;
(vii) a surfactant;
(viii) an alkanoic acid;
(ix) a lactam compound;
(x) an alkanol; and
(xi) a dialkylamino acetate.

14. The method of claim 12, further comprising an emulsifying agent selected from the group comprising:
(i) cera alba;
(ii) cera flava;
(iii) carbomer;
(iv) glycerol stearate;
(v) emulsifying wax NF;
(vi) cetaryl alcohol;
(vii) PEG-20 stearate;
(viii) cetyl alcohol;
(ix) propylene glycol;
(x) stearyl alcohol NF;
(xi) polysorbate 80; and
(xii) lecithin.

15. The method of claim 12, further comprising a skin conditioner selected from the group comprising:
(i) an emollient;
(ii) a humectants;
(iii) an emulsifier;
(iv) a preservative;
(v) a skin aid;
(vi) a conditioner;
(vii) a chelating agent;
(viii) a rheology modifier;
(ix) a moisturizer;
(x) a scent; and
(xi) a diluent.

16. The method of claim 12, further comprising a first essential oil within an effective range from about 500 ppm to about 3500 ppm.

17. The method of claim 16, further comprising a second essential oil within an effective range from about 500 ppm to about 3500 ppm.

18. A composition for treating a dermatophytic infection, comprising:
an excipient;
a penetration enhancer; and
at least one antifungal essential oil components selected from the group comprising:
(vii) cinnamaldehyde
(viii) juniper
(ix) thymol
(x) citral
(xi) eugenol
(xii) carvacrol.

19. The composition of claim 18, further comprising a skin conditioner selected from the group comprising:
(i) an emollient;
(ii) a humectants;
(iii) an emulsifier;
(iv) a preservative;
(v) a skin aid;
(vi) a conditioner;
(vii) a chelating agent;
(viii) a rheology modifier;
(ix) a moisturizer;
(x) a scent; and
(xi) a diluent.

20. The composition of claim 18, wherein the penetration enhancer is selected from the group comprising:
(i) isopropyl myristate;
(ii) an alcohol;
(iii) an n-alkanol;
(iv) a sulfoxide;
(v) an ester;
(vi) an amide;
(vii) a surfactant;
(viii) an alkanoic acid;
(ix) a lactam compound;
(x) an alkanol; and
(xi) a dialkylamino acetate.
